(19) United States
(12) Patent Application Publication
(10) Pub. No.: US 2003/0077636 A1
(43) Pub. Date: Apr. 24, 2003

(54) Use of particles which have signal-emitting characteristics and at least one group with an affinity for maked nucleic acids

(76) Inventors: Andreas Bosio, (US); Alexander Scheffold, Berlin (DE)

Correspondence Address: JACOBSON HOLMAN PLLC, 400 SEVENTH STREET N.W., SUITE 600, WASHINGTON, DC, 20004, US

(21) Appl. No.: 10/239,424

(22) PCT Filed: Mar. 31, 2001
(86) PCT No.: PCT/EP01/03702

(30) Foreign Application Priority Data
Mar. 31, 2000 (DE) 100 16 115.4
Jun. 27, 2000 (EP) 00113549.0

(51) Int. Cl. C12Q 1/68

(52) U.S. Cl. 435/6

(57) ABSTRACT

The Use of particles with signal-emitting characteristics and at least one group with an affinity for labeled nucleic acids for the detection of labeled nucleic acids which are hybridized with nucleic acids immobilized on a surface. Another subject matter of the invention are supports with particles with signal-emitting characteristics and at least one group with an affinity for labeled nucleic acids for the detection of labeled nucleic acids.

The subject matter of the present invention are the use according to claim 1 and the support according to claim 8.

The invention pertains to a support which enables to bind two or more differently labeled nucleic acids which have been hybridized independently from each other with nucleic acids immobilized on a surface and render them measurable using the different signal-emitting molecules incorporated into the support or provided thereon.

Microarrays (solid supports having nucleic acids immobilized thereon at different addressable positions) are increasingly used for the parallel identification and the preparation of transcription profiles of polynucleic acids. Here, for hybridization the nucleic acids to be analyzed are modified such that they are afterwards identifiable and quantifiable (radioactivity, chemiluminescence, fluorescence, etc.). The modification may be either a molecule which can release a signal by itself (direct labeling) or a molecule which is recognized by a second molecule which in its turn is labeled (indirect labeling). A problem with the use of microarrays excluding a sensitive detection is the sensitivity with which the nucleic acids to be analyzed on said supports can be recognized and limited starting amounts of nucleic acid mixtures to be analyzed.

According to the state of the art there are two possibilities to solve this problem. Firstly, one tries to amplify the available nucleic acid in different ways before hybridizing it with the microarray (T7 in vitro transcription (Eberwine et al.) or PCR, to mention only two methods). Secondly, one tries to the recognize the already hybridized and chemically modified nucleic acids using indirect labeling and direct labeling in combination therewith and to create strong signals by a secondary reaction in combination with the recognized position, e.g., by enzymatic reactions (keywords sandwich, tyramine, horseradish peroxidase, etc.). The drawbacks of this approach are that the used enzymatic methods often result in a non-linear amplification, i.e., the amount of the existing different nucleic acids is amplified in a different manner. However, since the amount of nucleic acid is to be determined by microarray analysis, the non-uniform amplification questions the complete analysis. Furthermore, both enzymatic and non-enzymatic amplification reactions are often multistage reactions and very complex.

The object of the invention is to avoid the above-mentioned drawbacks and to ensure a more precise quantification of nucleic acids.

According to the invention, the addressed object is achieved by a use of particles selected from the group consisting of liposomes and/or micelles with signal-emitting characteristics and at least one group with an affinity for labeled nucleic acids for the detection of labeled nucleic acids hybridized with a nucleic acid immobilized on a surface.

In a favorable embodiment of the use of the invention the nucleic acids bear different labels. This enables, e.g., a differentiation between healthy and diseased tissue.

The particles preferably have a particle size from 20 to 4000 nm, preferably from 200 to 400 nm. The particles and the preparation thereof are described in Scheffold A, Miltenyi S, Radbruch A, Magnetofluorescent liposomes for increased sensitivity of immunofluorescence. Immunotechnology 1995; 1(2): 127-37.

The signal-emitting property is produced in particular by electromagnetic radiation, in particular fluorescence, radioactivity, luminiscence, phosphorescence, electromagnetic resonance.

The affinity group is preferably an antibody or an antibody fragment. The affinity group may be directed against any type of low-molecular haptens such as, e.g., Alexa Fluor 488; amino-3-deoxydigoxigenin hemisuccinamide; biotin; BODIPY, Cascade Blue; Cy2; Cy3; Cy5; Danisyl; DNP (dinitrophenol); DIG (digoxigenin); Alexa Fluor 488; 5(6)-SFX; fluorescein; Lucifer yellow iodoacetamide; Marina Blue; Oregon Green 488; 5(6)-TAMRA; PE (phycoerythrin); Rhodamine Red; Texas Red. The nucleic acids have to be labeled with the appropriate haptens.

Another object of the invention are supports having a surface on which nucleic acids hybridized with labeled nucleic acids are immobilized, where particles having signal-emitting properties and at least one group with an affinity for the labeled nucleic acids, accordingly the first affinity group, are bonded to the labeled nucleic acids.

The particles for the supports of the invention designated for the highly sensitive immunofluorescence detection of hybridized nucleic acids preferably consist of magnetofluorescent liposomes containing several thousand fluorescing molecules and colloidal magnetic particles.

The liposomes preferably consist of from 40 to 50 mol %, in particular 45 mol % of phosphatidylcholine, from 5 to 15 mol %, in particular 10 mol % of phosphatidylglycerol, from 2 to 10 mol %, in particular 5 mol % of phosphatidylethanolamine, and from 35 to 45 mol %, in particular 40 mol % of cholesterol.

When using water-soluble fluorophores (for example carboxyfluorescein), as a function of the solubility and the fluorescence behavior of the respective fluorophor the liposomes contain a 1 to 100 mM solution of the fluorescent dye which can be obtained by immersing the liposomes into a suitably concentrated fluorophor solution and a fluorophor diffusion into the liposomes.

When using water-insoluble fluorophores, the liposomes contain the respective fluorophore in a molar ratio of from 1:100 to 1:1000 as a function of the solubility and the fluorescence behavior of the respective fluorophore. The fluorophore-containing liposomes are obtained during the liposome preparation as a function of the solubility and the fluorescence behavior of the fluorophor by adding the respective fluorophore in a molar ratio of from 1:100 to 1:1000, based on the number of mols of the lipids, to the lipid mixture.

The liposomes contain superparamagnetic microparticles having diameters from 50 to 100 nm. Said particles are obtained by immersing the liposomes into a solution of a superparamagnetic microparticle with an absorption at 450 nm (optical density, OD₄₅₀) of 500.

A distinct difference between liposomes (particles having a pseudofluid, diphase surface) and all other microparticles is that due to fluid properties an affinity group is completely mobile on the liposome surface. This is of essential significance for the affinity or avidity (total bond strength between two molecules or particles which is influenced by several bond regions) of the particles since:

A) for theoretical, sterical and production-engineering reasons only a finite number of affinity groups can be provided on the surface of a particle;

B) the density of the applied affinity groups has to be selected very precisely since with the density being too high a mutual repulsion and hindrance is to be expected whereas with a too low loading density the probability of a bond to the target molecule is strongly reduced.

Thus, for sterical reasons the bond strength between rigid, spherical particles (microspheres) having diameters from 20 to 4000 nm and a relatively rigid tube having a diameter of about 2 nm (double-stranded DNA) is substantially determined by only one affinity group. However, the bond strength between fluid, spherical particles (liposomes and micelles) having diameters from 20 to 4000 nm and a relatively rigid tube having a diameter of about 2 nm (double-stranded DNA) is determined with the greatest probability by more than one affinity group. This may be explained as follows: after the bonding of a target molecule by a first affinity group is effected, additional affinity groups of the same liposome can migrate to a adjacent target molecule and effect a second bonding due to the constant lateral diffusion (about 10⁻⁶ to 10⁻⁷ m/s) which is perfectly well-known for lipid bilayers.

In first approximation, an antigen (target molecule) is bonded by one of both bonding sites of an antibody in a reversible bimolecular reaction. For most antibodies, the affinity constant K=10⁵ to 10¹¹ L/mol. The simple bimolecular reaction may only be applied in the reaction of an antigen having an epitope and homogeneous antibodies. If two identical epitopes exist on the antigen in a distance such that both bonding sites of an antibody or two connected antibodies (e.g., IgM or the liposomes provided with antibodies described here) react with the same or two adjacent, connected antigen molecules (labeled, double-stranded DNA), the reaction no longer proceeds in a bimolecular manner necessitating complicated equations to be applied on the kinetics of the immune complex formation. If both antibody bonding sites can react, e.g., the second bonding site bonds to the epitope much faster than the first one (entropic effect) since the antigen molecule is already very close to the antibody resulting in an increase of the apparent affinity (avidity) by a factor of at least 10⁴.

The increased affinity (avidity) results in an increased and more specific bonding which explains the higher sensitivity and specificity of liposomes, i.e. particles having a diphase, fluid surface, to other spheric particles.

Antibodies, e.g., anti DNP, anti DIG etc., were conjugated to the amino groups of phosphatidyl ethanolamine existing in liposomes using succinimidyl-4-(N-maleimidomethyl)cyclohexane-1-carboxylate by thiol groups which may be exposed by reagents reducing S-S groups. Typical reaction conditions were adjusted with dithiothreitol.

The RNAs to be tested were transcribed into the corresponding cDNA by a conventional reverse transcriptase (RT) reaction. Here, derivatized nucleotides such as DNP-dCTP, DIG-CTP, etc., which are incorporated into the formed cDNA, are added. Using nucleotides derivatized in a different manner, different cDNAs may be labeled in a different way in independent reactions. For hybridization said whole cDNAs labeled in a different manner, e.g., of healthy and diseased tissue, are applied to a microarray. After hybridization, the derivatized and hybridized cDNAs can be recognized using antibodies directed against them and conjugated with the liposomes and identified and quantified by measuring the different fluorescences. Hence, avoiding enzymatic amplification several thousand fluorescent molecules can be used for a correspondingly high sensitivity of the analysis in one step.

The invention will be illustrated in more detail by the following examples.

### EXAMPLE 1

### LIPSOMES FOR SIGNAL AMPLIFICATION

cDNA fragments having a length of about 300 bp were generated with incorporating biotin dUTP and digoxigenin dUTP, resp., by PCR (“EC20 biotin” and “EC20 digoxigenin”, resp.), dropped twice on a coated glass slide (300 pg) and covalently bonded (see PCT/EP 99/04014). Each spot on these “EC20Bio₁₃ EC20Dig arrays” contained a mixture of both cDNA fragments in the ratios EC20:EC20 digoxigenin of 1000:1; 200:1; 40:1; 8:1; 1:1; 1:8; 1:40; 1:200, 1:1000.

In addition, two negative controls were dropped on: 1) spotting buffer 2) herring semen DNA. The arrays were washed in TNT buffer at room temperature for 15 min, blocked with TNB buffer at room temperature for 30 min and subsequently dyed with Cy3 and Cy5 labeled liposomes (antiBio-Cy3 and antiDig-Cy5 liposomes, resp.) or with Cy3 labeled streptavidin (Strept Cy3) at room temperature with shaking (150 rpm) in a total volume of 50 μl. For this, the liposomes were washed before with the 10-fold PBS volume and centrifuged with 13,000 rpm at 4° C. for 20 min.

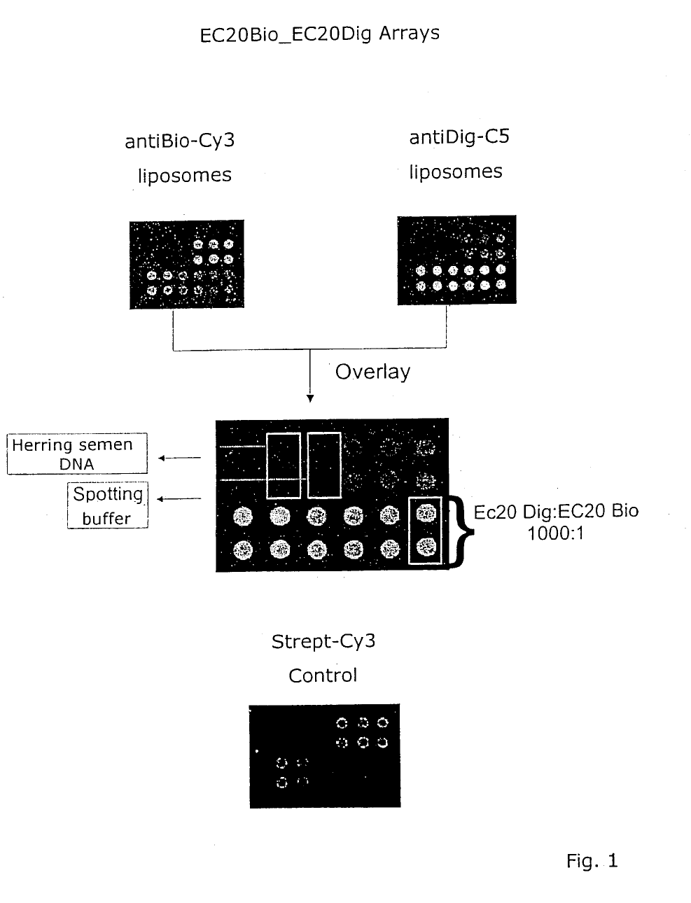

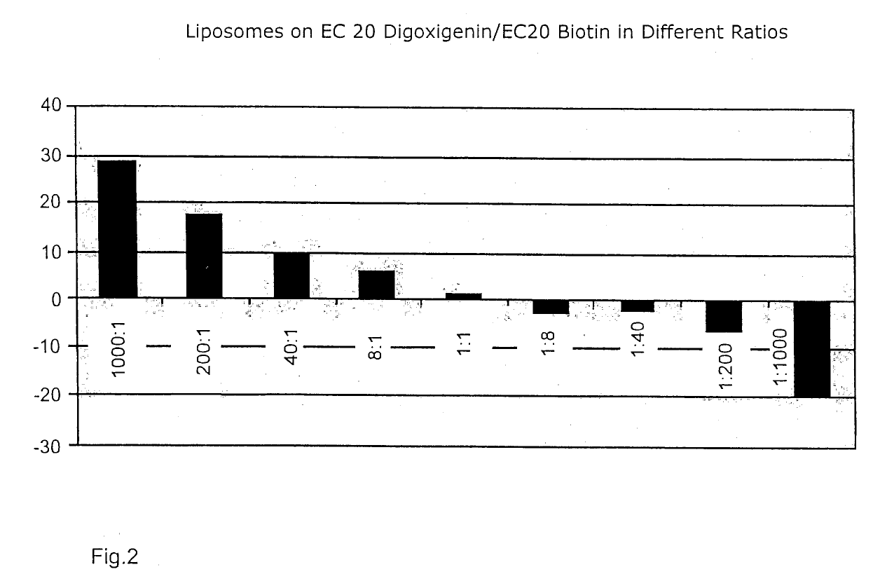

Subsequently, the dyed arrays were washed with shaking with TNT buffer for 20 min and with 0.06×SSC for 2 min. The arrays were analyzed in a laser scanner (ScanArray 3000, General Scanning, Inc.) and the signal intensities were calculated (Imagene 2.0, Biodiscovery, Inc.), see FIG. 1 and FIG. 2. In the combining figure (overlay) the green color represents bonded anti-Dig-Cy5 liposomes, whereas the red color represents anti biotin-Cy3 liposomes. It can be seen that a) the coloration of cDNA fragments is specific, b) the mixing ratios of the applied cDNA fragments are detectable and c) the signal intensity of the antiBio Cy3 liposomes is greater than that if Strep Cy3.

Example 2) This experiment intended to establish whether the coloration by means of liposomes results in a signal increase as compared to the conventional direct incorporation of fluorescence labeled nucleotides.

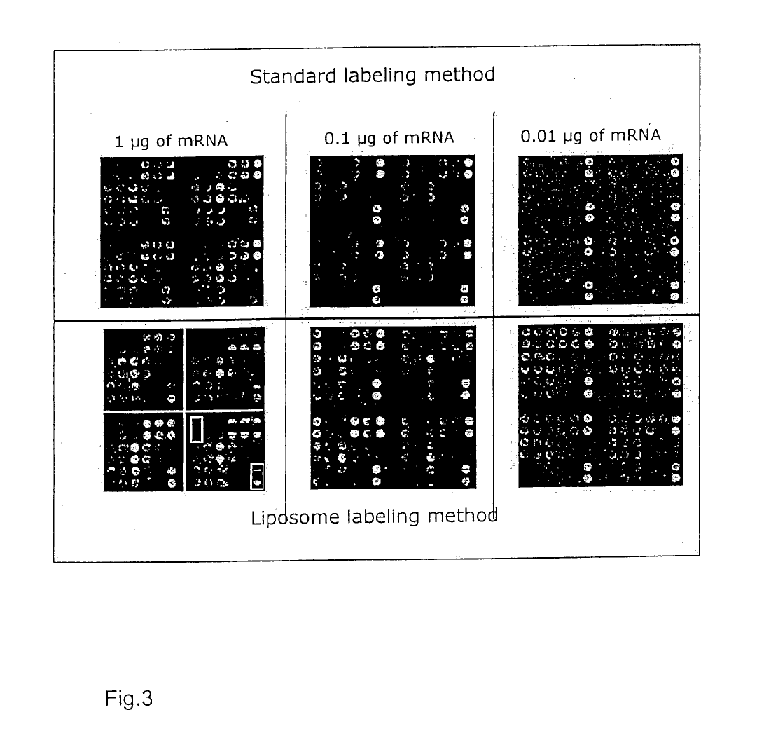

For this, cDNA arrays with 18 different cDNAs (300 pg) were prepared with each cDNA being applied 8 times in total (four identical quandrants with 2 spots each). The applied cDNAs are summarized in table 1. The cDNA sequence in the respective quadrants is from the bottom of the right hand side to the top of the left hand side such that, e.g., cDNA no. 1 “EC7” can be found in the bottom right hand corner of the respective quadrant. For the test RNA was extracted from murine spleen and murine liver. From this mRNA was isolated. 1 μg, 0.1 μg and 0.01 μg, resp., of mRNA were transcribed into cDNA in a reverse transcription with incorporating a) fluorescence labeled nucleotides (Cy3-dCTP for spleen and Cy5-dCTP for liver, resp.) and b) biotinylated (spleen) and digoxigenated (liver) dUTP. Each of the conventionally labeled Cy3 and Cy5 cDNAs were hybridized together on an array. FIG. 3 reveals that the signal intensity decreases with a decrease of the initial amount such that at an initial amount of 0.01 μg signals are rarely identifiable. The samples labeled with biotin and digoxigenin, resp., were also hybridized together on arrays and subsequently labeled with anti-Bio-Cy3 and antiDig-Cy5 liposomes, resp., as described in example 1. Here, it can be recognized that the signal intensity also decreases with a decrease of the initial amount. However, also at 0.01 μg all signals are identifiable. This shows that the liposome labeling is much more sensitive than the conventional direct labeling.

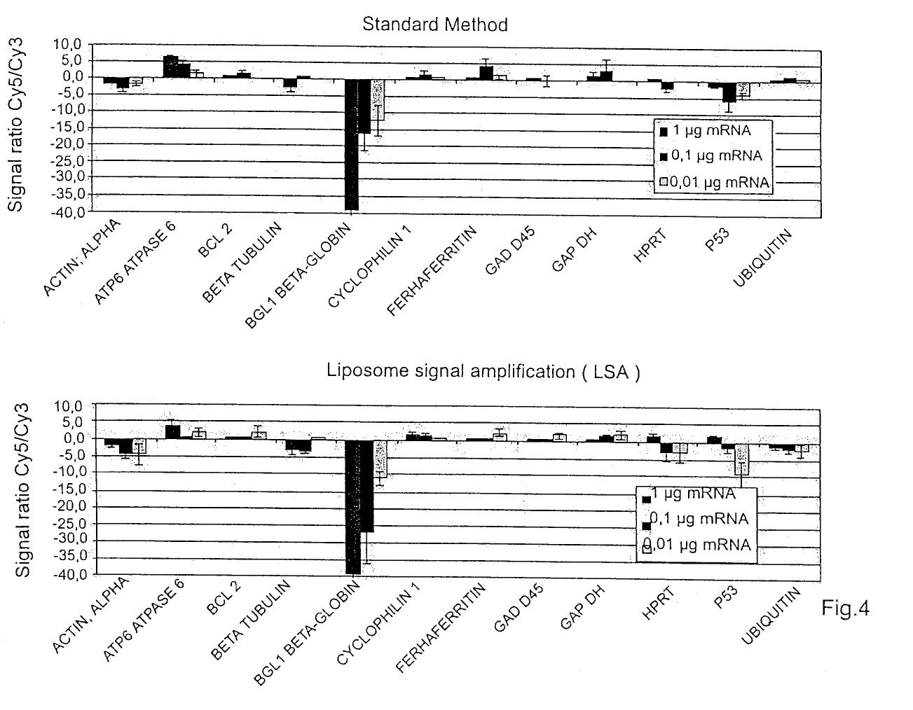

FIG. 4 summarizes the signal quotients of the single arrays in a bar graph. Here, it can be recognized that the signal quotients for both methods are comparable except that with the standard method the yellow bars (initial amount of 0.01 μg) are not present for some genes since the absolute signal intensity of the basic signals is too low. Table 2 again summarizes the signal quotients in tabular form. In addition, the number of detectable genes is illustrated here. Those genes resulting in signal intensities in at least one of both fluorescence canals being at least two times greater than the corresponding signal intensity of the mean value of the negative controls (herring semen DNA and salt) are termed as “detectable genes”. Again, this demonstrates that the liposome labeling is more sensitive than the conventional direct labeling.

TABLE 1

| No. | Gene Name |
|---|---|
| 1 | Ec7 |
| 2 | Salt |
| 3 | Salmsperm DNA |
| 4 | GAPDH |
| 5 | HPRT |
| 6 | Ec17 |
| 7 | GAD D45 |
| 8 | beta Tubulin |
| 9 | Actin; alpha |
| 10 | Ubiquitin |
| 11 | Cyclophilin 1 |
| 12 | BGL1 Beta-globin |
| 13 | Ec20 |
| 14 | FERHA ferritin heavy chain |
| 15 | AT Pase 6 |
| 16 | Bcl2 |
| 17 | p53 |
| 18 | Ec21 |

TABLE 2

| | 1 μg mRNA | | 1 μg RNA | |
|---|---|---|---|---|
| | starting mat | | starting mat | |
| Name | Liposome | Standard | Liposome | Standard |
| ACTIN, ALPHA | −1.9 | −1.3 | −4.2 | −1.7 |
| ATP6 ATPASE 6 | 4.2 | 6.3 | 1.0 | 1.5 |
| BCL2 | 1.0 | 1.0 | 1.0 | n.d. |
| BETA TUBULIN | −3.0 | −2.6 | −3.3 | n.d. |
| BGL1 BETA-GLOBIN | −39.3 | −39.0 | −27.1 | 12.1 |
| CYCLOPHILIN 1 | 1.5 | 1.0 | 1.3 | 1.0 |
| FERHA FERRITIN | 1.0 | 1.0 | 1.0 | 1.3 |
| GAD D45 | 1.0 | 1.0 | 1.0 | n.d. |
| GAP DH | 1.0 | 1.9 | 1.6 | n.d. |
| HPRT | 1.5 | 1.0 | −2.8 | n.d. |
| P53 | 1.6 | −1.1 | −1.6 | −4.0 |
| UBIQUITIN | −1.3 | 1.0 | −1.9 | 1.0 |
| Detectable genes | 12 | 12 | 12 | 7 |
| (max. 12) | | | | |

1. Use of particles selected from the group consisting of liposomes and/or micelles with signal-emitting characteristics and at least one group with an affinity for labeled nucleic acids for the detection of labeled nucleic acids which are hybridized with nucleic acids immobilized on a surface.

2. The use according to claim 1, wherein the particles bear different affinity groups and/or have different signal-emitting properties.

3. The use according to any one or more of claims 1 or 2, wherein the particles have a particle size from 20 to 4000 nm.

4. The use according to any one or more of claims 1 to 3, wherein the signal-emitting property bases on electromagnetic radiation, in particular fluorescence, radioactivity, luminescence, phosphorescence, electromagnetic resonance or combinations thereof.

5. The use according to any one or more of claims 1 to 4, wherein the affinity group is an antibody or an antibody fragment.

6. The use according to any one or more of claims 1 to 5, wherein the affinity group is directed against low-molecular haptens.

7. The use according to any one or more of claims 1 to 6, wherein the nucleic acids are labeled with low-molecular haptens.

8. A support with a surface on which nucleic acids hybridized with labeled nucleic acids are immobilized, wherein particles with signal-emitting properties and at least one group with an affinity for the labeled nucleic acids, thereafter the first affinity group, are bonded to the labeled nucleic acids.

9. The support according to claim 8, wherein the particles bear different affinity groups and/or have different signal-emitting properties.
